(19) United States
(12) Patent Application Publication
(10) Pub. No.: US 2021/0331709 A1
(43) Pub. Date: Oct. 28, 2021

(54) VEHICLE CONTROL DEVICE AND CONTROL METHOD FOR THE SAME

(71) Applicant: LG ELECTRONICS INC., Seoul (KR)

(72) Inventors: Hyongguk KIM, Seoul (KR); Hyeongjin IM, Seoul (KR); Jaehoon CHO, Seoul (KR); Heejeong HEO, Seoul (KR); Yoonjung HONG, Seoul (KR)

(73) Assignee: LG ELECTRONICS INC., Seoul (KR)

(21) Appl. No.: 16/490,065

(22) PCT Filed: Apr. 19, 2019
(86) PCT No.: PCT/KR2019/004754

§ 371 Date: Aug. 29, 2019

(51) Int. Cl. B60W 60/00; B60W 50/14; A61B 5/00

(52) U.S. Cl. CPC B60W 60/0051; B60W 50/14; B60W 2050/0026; B60W 2540/22; A61B 5/6893

(57) ABSTRACT

The present invention relates to a vehicle capable of driving autonomously and a method of recommending a more appropriate driving mode depending on a driver's status. A vehicle control device for controlling a vehicle comprises: a memory that stores a driving stress map containing stress level information which is calculated for each road section based on a driver's stress information collected while the vehicle is driving on each road section; and a processor that retrieves a stress level for a road section where the vehicle is currently located from the driving stress map and outputs notification information recommending a change to a first driving mode or second driving mode according to the retrieved stress level. The vehicle may perform autonomous driving by the vehicle control apparatus.

### TECHNICAL FIELD

The present invention relates to a vehicle capable of driving autonomously and a method of recommending a more appropriate driving mode depending on a driver's status.

### BACKGROUND ART

A vehicle is an apparatus capable of moving a user in the user-desired direction. Typically, a representative example may be a car.

Meanwhile, for convenience of a user using a vehicle, various types of sensors and electronic devices are provided in the vehicle. Specifically, a study on an Advanced Driver Assistance System (ADAS) is actively undergoing. In addition, an autonomous vehicle is actively under development.

A vehicle may be included as a means of transportation. The means of transportation may refer to a means used to transport people or cargo. Examples of this may include cars, motorcycles, bicycles, trains, buses, and trams. What is described in relation to vehicles in this specification may apply by analogy equally or similarly to all means of transportation.

These days, research related to artificial intelligence (AI) is actively being carried out. Also, there is ongoing research on vehicles combined with artificial intelligence which are more convenient for users to use. Some types of vehicles, such as autonomous vehicles, are emerging as part of this research.

As part of the artificial intelligence research, active research is going on to acquire a driver's biometric information and provide various functions based on the acquired biometric information. Also, as part of this research, research is being conducted on functions for acquiring a driver's biometric information, detecting a deterioration in the driver's health condition or a sudden abnormality in their health, and urging the driver to take a rest or giving aid to the driver through emergency calls according to a detection result.

Furthermore, as part of this research, active research is going on to prevent any deterioration in the driver's heath condition or any abnormality in their health while driving.

### DISCLOSURE

### Technical Problem

Therefore, an object of the present invention is to provide a vehicle control device capable of detecting a road section where a driver is under a lot of stress and allowing for autonomous driving on the detected road section and a control method for the vehicle control device.

Another object of the present invention is to provide a vehicle control device capable of providing a driving mode suitable for a driver on each road section based on information about the driver's physical condition collected on each road section and a control method for the vehicle control device.

### Technical Solution

An exemplary embodiment of the present invention provides a vehicle control device for controlling a vehicle, including: a memory that stores a driving stress map containing stress level information which is calculated for each road section based on a driver's stress information collected while the vehicle is driving on each road section; and a processor that retrieves a stress level for a road section where the vehicle is currently located from the driving stress map and outputs notification information recommending a change to a first driving mode or second driving mode according to the retrieved stress level.

The processor controls the vehicle to output first notification information recommending a change to the first driving mode or second driving mode, based on whether the stress level for the current location of the vehicle exceeds a preset, first value.

If the stress level for the current location of the vehicle exceeds the preset, first reference value and also exceeds a second reference value, which is higher than the first reference value, the processor controls the vehicle to output second notification information indicating an automatic change to the first driving mode, and, if the stress level for the current location of the vehicle is equal to or lower than the first reference value and lower than a third reference value, which is lower than the first reference value, the processor controls the vehicle to output third notification information indicating an automatic change to the second driving mode.

The processor collects the stress information such as the driver's biometric information acquired for the road section the vehicle is currently driving on and information related to the driver's specific behavior detected while the vehicle is driving.

The processor detects whether the vehicle enters a second road section which is different from a first road section the vehicle is currently driving on, and calculates a stress score from stress information collected for the first road section according to the detection result and updates an existing stress level calculated for the first road section.

If the vehicle enters a handover zone set for the first road section, the processor detects that the vehicle is entering the second road section, retrieves a stress level for the second road section from the driving stress map, and outputs the notification information according to the retrieved stress level.

The processor varies the distance of the handover zone based on a driving mode suitable for the stress level for the second road section and the driving speed of the vehicle.

The processor controls the vehicle to alter a function of collecting and displaying information on situations around the vehicle based on the stress level for the road section where the vehicle is currently located, the stress level being retrieved from the driving stress map.

The processor controls the vehicle to change the picture quality of a black box or the resolution of captured images based on the retrieved stress level or to change the strength or exchange cycle of communication signals for V2X (vehicle-to-things) or V2V (vehicle-to-vehicle).

If the retrieved stress level is higher than a preset level, the processor controls the vehicle to display road situation information collected from around the vehicle, in place of dashboard information outputted through CIDs (central information displays).

The processor calculates the ratio of autonomous vehicles and manually driven vehicles to other vehicles located within a preset range from the vehicle, and, if the calculation result shows that the ratio of vehicles driving in a specific driving mode is equal to or higher than a preset value, compares the specific driving mode and the driving mode of the vehicle and controls the vehicle to output notification information recommending a driving mode change to the specific driving mode according to the comparison result.

The processor controls the vehicle to output the notification information according to a result of comparing a driving mode suitable for the stress level for the road section where the vehicle is currently located and the current driving mode of the vehicle, the stress level being retrieved from the driving stress map, and the current driving mode of the vehicle.

When the vehicle is driving in manual driving mode, the processor controls the vehicle to output notification information recommending a change to autonomous driving mode based on a result of sensing the driver's biometric information.

When the vehicle is driving in manual driving mode, the processor controls the vehicle in such a way that the driver is forced to switch to autonomous driving mode and drive around an object detected from around the vehicle based on a result of sensing the driver's biometric information and the possibility of colliding the object.

When the vehicle is driving in manual driving mode, the processor controls the vehicle in such a way that at least one of the vehicle's functions is restricted based on a result of sensing the driver's biometric information, wherein the restricted vehicle function involves speeding up to over a certain speed and changing lanes.

Another exemplary embodiment of the present invention provides a control method for a vehicle control device for controlling a vehicle, the control method including: a first step of retrieving a stress level for a road section the vehicle is driving on from a driving stress map, the driving stress map containing stress level information which is calculated for each road section based on a driver's stress information collected while the vehicle is driving on each road section; a second step of determining whether a driving mode suitable for the road section the vehicle is currently driving on is autonomous driving mode or manual driving mode, based on the retrieved stress level; a third step of determining whether an automatic change to the driving mode determined in the second step is necessary, based on the retrieved stress level; and a fourth step of outputting notification information recommending a change to a specific driving mode or notification information indicating a change to the specific driving mode, according to the result of the determination in the third step.

### Advantageous Effect

Embodiments of the present invention provide one or more advantages as follows.

A vehicle according to an embodiment of the present invention has the advantage of reducing a driver's stress while driving the vehicle by switching the driving mode to autonomous driving mode, if the vehicle is currently driving in a road section where the driver is usually under a lot of stress.

A vehicle according to an embodiment of the present invention has the advantage of preventing an accident or an abnormality in the driver's health condition by checking the driver's biological information and performing autonomous driving according to the check result.

### BRIEF DESCRIPTION OF THE DRAWINGS

FIG. 1 is a view showing the exterior appearance of a vehicle in accordance with an embodiment of the present invention.

FIG. 2 is a view showing a vehicle in accordance with an embodiment of the present invention when viewed from the outside from different angles.

FIGS. 3 and 4 are views showing the interior of a vehicle in accordance with an embodiment of the present invention.

FIGS. 5 and 6 are reference views illustrating objects in accordance with an embodiment of the present invention.

FIG. 7 is a block diagram illustrating a vehicle in accordance with an embodiment of the present invention.

FIG. 8 is a flowchart showing an operational process for recommending a driving mode suitable for a current road section, in a vehicle in accordance with an embodiment of the present invention.

FIG. 9 is a flowchart showing an operational process for updating a stress level in a driving stress map based on stress information collected while driving, in a vehicle in accordance with an embodiment of the present invention.

FIG. 10 is a flowchart showing an operational process for setting a handover zone for a current road section, in a vehicle in accordance with an embodiment of the present invention.

FIG. 11 is an illustration showing an example of collecting stress information from a driver and an example of a driving stress map to which calculated stress levels are mapped, in a vehicle in accordance with an embodiment of the present invention.

FIG. 12 is an illustration showing an example of notification information recommending a driver to switch to autonomous driving mode or indicating an automatic change to autonomous driving mode, in a vehicle in accordance with an embodiment of the present invention.

### MODES FOR CARRYING OUT THE PREFERRED EMBODIMENTS

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In describing the present disclosure, if a detailed explanation for a related known function or construction is considered to unnecessarily divert the gist of the present disclosure, such explanation has been omitted but would be understood by those skilled in the art. The accompanying drawings are used to help easily understand the technical idea of the present disclosure and it should be understood that the idea of the present disclosure is not limited by the accompanying drawings. The idea of the present disclosure should be construed to extend to any alterations, equivalents and substitutes besides the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be connected with the another element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

A vehicle according to an embodiment of the present invention may be understood as a conception including cars, motorcycles and the like. Hereinafter, the vehicle will be described based on a car.

The vehicle according to the embodiment of the present invention may be a conception including all of an internal combustion engine car having an engine as a power source, a hybrid vehicle having an engine and an electric motor as power sources, an electric vehicle having an electric motor as a power source, and the like.

In the following description, a left side of a vehicle refers to a left side in a driving direction of the vehicle, and a right side of the vehicle refers to a right side in the driving direction.

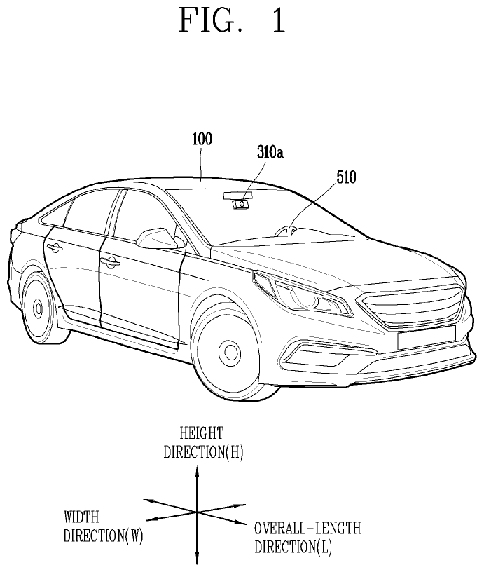

FIG. 1 is a view illustrating appearance of a vehicle in accordance with an embodiment of the present invention.

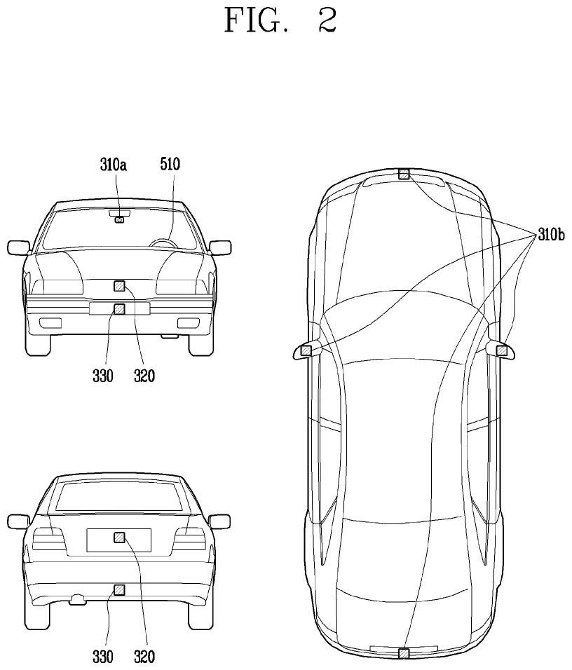

FIG. 2 is a view illustrating appearance of a vehicle at various angles in accordance with an embodiment of the present invention.

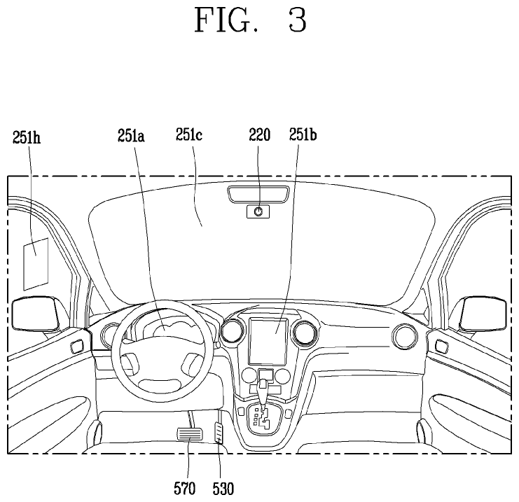

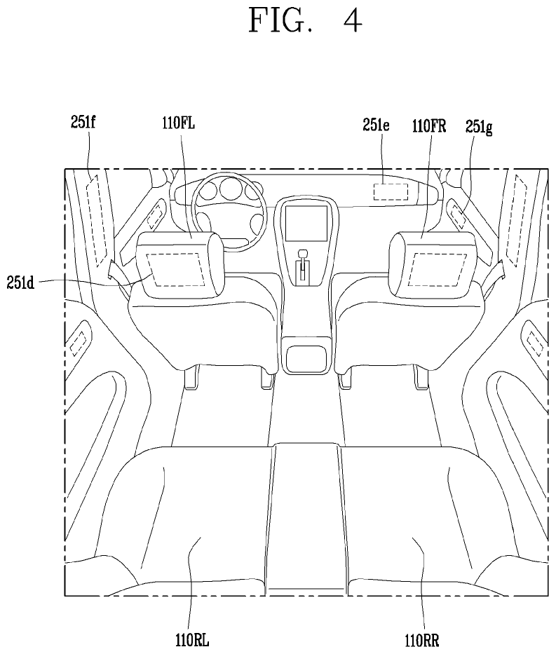

FIGS. 3 and 4 are views illustrating an inside of a vehicle in accordance with an embodiment of the present invention.

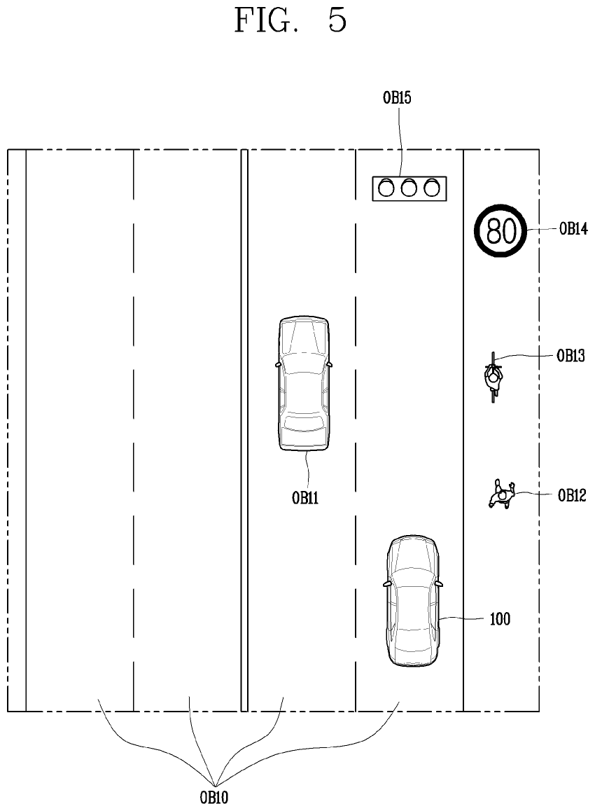

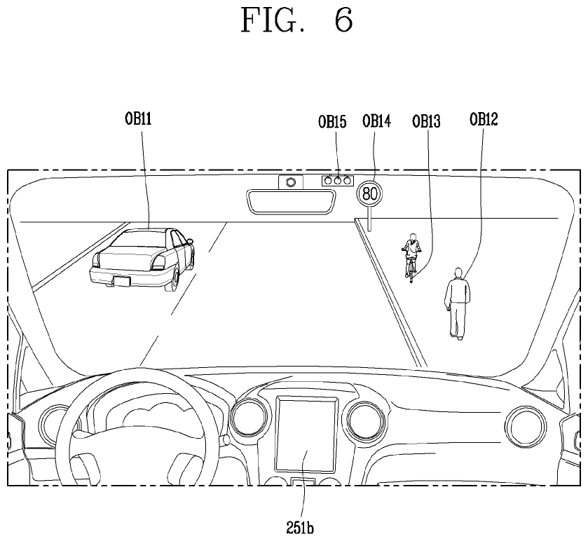

FIGS. 5 and 6 are reference views illustrating objects in accordance with an embodiment of the present invention.

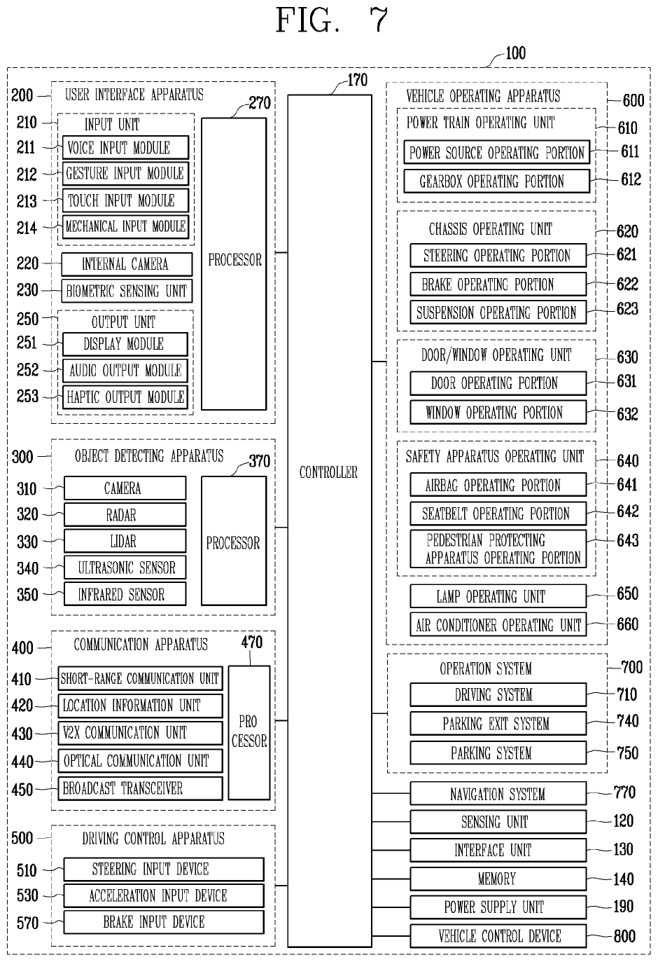

FIG. 7 is a block diagram illustrating a vehicle in accordance with an embodiment of the present invention.

As illustrated in FIGS. 1 to 7, a vehicle 100 may include wheels turning by a driving force, and a steering apparatus 510 for adjusting a driving (ongoing, moving) direction of the vehicle 100.

The vehicle 100 may be an autonomous vehicle.

The vehicle 100 may be switched into an autonomous mode or a manual mode based on a user input.

For example, the vehicle may be converted from the manual mode into the autonomous mode or from the autonomous mode into the manual mode based on a user input received through a user interface apparatus 200.

The vehicle 100 may be switched into the autonomous mode or the manual mode based on driving environment information. The driving environment information may be generated based on object information provided from an object detecting apparatus 300.

For example, the vehicle 100 may be switched from the manual mode into the autonomous mode or from the autonomous module into the manual mode based on driving environment information generated in the object detecting apparatus 300.

In an example, the vehicle 100 may be switched from the manual mode into the autonomous mode or from the autonomous module into the manual mode based on driving environment information received through a communication apparatus 400.

The vehicle 100 may be switched from the manual mode into the autonomous mode or from the autonomous module into the manual mode based on information, data or signal provided from an external device.

When the vehicle 100 is driven in the autonomous mode, the autonomous vehicle 100 may be driven based on an operation system 700.

For example, the autonomous vehicle 100 may be driven based on information, data or signal generated in a driving system 710, a parking exit system 740 and a parking system 750.

When the vehicle 100 is driven in the manual mode, the autonomous vehicle 100 may receive a user input for driving through a driving control apparatus 500. The vehicle 100 may be driven based on the user input received through the driving control apparatus 500.

An overall length refers to a length from a front end to a rear end of the vehicle 100, a width refers to a width of the vehicle 100, and a height refers to a length from a bottom of a wheel to a roof. In the following description, an overall-length direction L may refer to a direction which is a criterion for measuring the overall length of the vehicle 100, a width direction W may refer to a direction that is a criterion for measuring a width of the vehicle 100, and a height direction H may refer to a direction that is a criterion for measuring a height of the vehicle 100.

As illustrated in FIG. 7, the vehicle 100 may include a user interface apparatus 200, an object detecting apparatus 300, a communication apparatus 400, a driving control apparatus 500, a vehicle operating apparatus 600, an operation system 700, a navigation system 770, a sensing unit 120, an interface unit 130, a memory 140, a controller 170 and a power supply unit 190.

According to embodiments, the vehicle 100 may include more components in addition to components to be explained in this specification or may not include some of those components to be explained in this specification.

The user interface apparatus 200 is an apparatus for communication between the vehicle 100 and a user. The user interface apparatus 200 may receive a user input and provide information generated in the vehicle 100 to the user. The vehicle 200 may implement user interfaces (UIs) or user experiences (UXs) through the user interface apparatus 200.

The user interface apparatus 200 may include an input unit 210, an internal camera 220, a biometric sensing unit 230, an output unit 250 and a processor 270.

According to embodiments, the user interface apparatus 200 may include more components in addition to components to be explained in this specification or may not include some of those components to be explained in this specification.

The input unit 200 may allow the user to input information. Data collected in the input unit 120 may be analyzed by the processor 270 and processed as a user's control command.

The input unit 200 may be disposed inside the vehicle. For example, the input unit 200 may be disposed on one area of a steering wheel, one area of an instrument panel, one area of a seat, one area of each pillar, one area of a door, one area of a center console, one area of a headlining, one area of a sun visor, one area of a wind shield, one area of a window or the like.

The input unit 200 may include a voice input module 211, a gesture input module 212, a touch input module 213, and a mechanical input module 214.

The audio input module 211 may convert a user's voice input into an electric signal. The converted electric signal may be provided to the processor 270 or the controller 170.

The voice input module 211 may include at least one microphone.

The gesture input module 212 may convert a user's gesture input into an electric signal. The converted electric signal may be provided to the processor 270 or the controller 170.

The gesture input module 212 may include at least one of an infrared sensor and an image sensor for detecting the user's gesture input.

According to embodiments, the gesture input module 212 may detect a user's three-dimensional (3D) gesture input. To this end, the gesture input module 212 may include a light emitting diode outputting a plurality of infrared rays or a plurality of image sensors.

The gesture input module 212 may detect the user's 3D gesture input by a time of flight (TOF) method, a structured light method or a disparity method.

The touch input module 213 may convert the user's touch input into an electric signal. The converted electric signal may be provided to the processor 270 or the controller 170.

The touch input module 213 may include a touch sensor for detecting the user's touch input.

According to an embodiment, the touch input module 213 may be integrated with the display module 251 so as to implement a touch screen. The touch screen may provide an input interface and an output interface between the vehicle 100 and the user.

The mechanical input module 214 may include at least one of a button, a dome switch, a jog wheel and a jog switch. An electric signal generated by the mechanical input module 214 may be provided to the processor 270 or the controller 170.

The mechanical input module 214 may be arranged on a steering wheel, a center fascia, a center console, a cockpit module, a door and the like.

The internal camera 220 may acquire an internal image of the vehicle. The processor 270 may detect a user's state based on the internal image of the vehicle. The processor 270 may acquire information related to the user's gaze from the internal image of the vehicle. The processor 270 may detect a user gesture from the internal image of the vehicle.

The biometric sensing unit 230 may acquire the user's biometric information. The biometric sensing module 230 may include a sensor for detecting the user's biometric information and acquire fingerprint information and heart rate information regarding the user using the sensor. The biometric information may be used for user authentication.

The output unit 250 may generate an output related to a visual, audible or tactile signal.

The output unit 250 may include at least one of a display module 251, an audio output module 252 and a haptic output module 253.

The display module 251 may output graphic objects corresponding to various types of information.

The display module 251 may include at least one of a liquid crystal display (LCD), a thin film transistor-LCD (TFT LCD), an organic light-emitting diode (OLED), a flexible display, a three-dimensional (3D) display and an e-ink display.

The display module 251 may be inter-layered or integrated with a touch input module 213 to implement a touch screen.

The display module 251 may be implemented as a head up display (HUD). When the display module 251 is implemented as the HUD, the display module 251 may be provided with a projecting module so as to output information through an image which is projected on a windshield or a window.

The display module 251 may include a transparent display. The transparent display may be attached to the windshield or the window.

The transparent display may have a predetermined degree of transparency and output a predetermined screen thereon. The transparent display may include at least one of a thin film electroluminescent (TFEL), a transparent OLED, a transparent LCD, a transmissive transparent display and a transparent LED display. The transparent display may have adjustable transparency.

Meanwhile, the user interface apparatus 200 may include a plurality of display modules 251a to 251g.

The display module 251 may be disposed on one area of a steering wheel, one area 521a, 251b, 251e of an instrument panel, one area 251d of a seat, one area 251f of each pillar, one area 251g of a door, one area of a center console, one area of a headlining or one area of a sun visor, or implemented on one area 251c of a windshield or one area 251h of a window.

The audio output module 252 converts an electric signal provided from the processor 270 or the controller 170 into an audio signal for output. To this end, the audio output module 252 may include at least one speaker.

The haptic output module 253 generates a tactile output. For example, the haptic output module 253 may vibrate the steering wheel, a safety belt, a seat 110FL, 110FR, 110RL, 110RR such that the user can recognize such output.

The processor 270 may control an overall operation of each unit of the user interface apparatus 200.

According to an embodiment, the user interface apparatus 200 may include a plurality of processors 270 or may not include any processor 270.

When the processor 270 is not included in the user interface apparatus 200, the user interface apparatus 200 may operate according to a control of a processor of another apparatus within the vehicle 100 or the controller 170.

Meanwhile, the user interface apparatus 200 may be called as a display apparatus for vehicle.

The user interface apparatus 200 may operate according to the control of the controller 170.

The object detecting apparatus 300 is an apparatus for detecting an object located at outside of the vehicle 100.

The object may be a variety of objects associated with driving (operation) of the vehicle 100.

Referring to FIGS. 5 and 6, an object O may include a traffic lane OB10, another vehicle OB11, a pedestrian OB12, a two-wheeled vehicle OB13, traffic signals OB14 and OB15, light, a road, a structure, a speed hump, a terrain, an animal and the like.

The lane OB01 may be a driving lane, a lane next to the driving lane or a lane on which another vehicle comes in an opposite direction to the vehicle 100. The lanes OB10 may be a concept including left and right lines forming a lane.

The another vehicle OB11 may be a vehicle which is moving around the vehicle 100. The another vehicle OB11 may be a vehicle located within a predetermined distance from the vehicle 100. For example, the another vehicle OB11 may be a vehicle which moves before or after the vehicle 100.

The pedestrian OB12 may be a person located near the vehicle 100. The pedestrian OB12 may be a person located within a predetermined distance from the vehicle 100. For example, the pedestrian OB12 may be a person located on a sidewalk or roadway.

The two-wheeled vehicle OB12 may refer to a vehicle (transportation facility) that is located near the vehicle 100 and moves using two wheels. The two-wheeled vehicle OB12 may be a vehicle that is located within a predetermined distance from the vehicle 100 and has two wheels. For example, the two-wheeled vehicle OB13 may be a motorcycle or a bicycle that is located on a sidewalk or roadway.

The traffic signals may include a traffic light OB15, a traffic sign OB14 and a pattern or text drawn on a road surface.

The light may be light emitted from a lamp provided on another vehicle. The light may be light generated from a streetlamp. The light may be solar light.

The road may include a road surface, a curve, an upward slope, a downward slope and the like.

The structure may be an object that is located near a road and fixed on the ground. For example, the structure may include a streetlamp, a roadside tree, a building, an electric pole, a traffic light, a bridge and the like.

The terrain may include a mountain, a hill and the like.

Meanwhile, objects may be classified into a moving object and a fixed object. For example, the moving object may be a concept including another vehicle and a pedestrian. The fixed object may be a concept including a traffic signal, a road and a structure, for example.

The object detecting apparatus 300 may include a camera 310, a radar 320, a LiDAR 330, an ultrasonic sensor 340, an infrared sensor 350 and a processor 370.

According to an embodiment, the object detecting apparatus 300 may further include other components in addition to the components described, or may not include some of the components described.

The camera 310 may be located on an appropriate portion outside the vehicle to acquire an external image of the vehicle. The camera 310 may be a mono camera, a stereo camera 310a, an around view monitoring (AVM) camera 310b or a 360-degree camera.

For example, the camera 310 may be disposed adjacent to a front windshield within the vehicle to acquire a front image of the vehicle. Or, the camera 310 may be disposed adjacent to a front bumper or a radiator grill.

For example, the camera 310 may be disposed adjacent to a rear glass within the vehicle to acquire a rear image of the vehicle. Or, the camera 310 may be disposed adjacent to a rear bumper, a trunk or a tail gate.

For example, the camera 310 may be disposed adjacent to at least one of side windows within the vehicle to acquire a side image of the vehicle. Or, the camera 310 may be disposed adjacent to a side mirror, a fender or a door.

The camera 310 may provide an acquired image to the processor 370.

The radar 320 may include electric wave transmitting and receiving portions. The radar 320 may be implemented as a pulse radar or a continuous wave radar according to a principle of emitting electric waves. The radar 320 may be implemented in a frequency modulated continuous wave (FMCW) manner or a frequency shift Keyong (FSK) manner according to a signal waveform, among the continuous wave radar methods.

The radar 320 may detect an object in a time of flight (TOF) manner or a phase-shift manner through the medium of the electric wave, and detect a position of the detected object, a distance from the detected object and a relative speed with the detected object.

The radar 320 may be disposed on an appropriate position outside the vehicle for detecting an object which is located at a front, rear or side of the vehicle.

The LiDAR 330 may include laser transmitting and receiving portions. The LiDAR 330 may be implemented in a time of flight (TOF) manner or a phase-shift manner.

The LiDAR 330 may be implemented as a drive type or a non-drive type.

For the drive type, the LiDAR 330 may be rotated by a motor and detect object near the vehicle 100.

For the non-drive type, the LiDAR 330 may detect, through light steering, objects which are located within a predetermined range based on the vehicle 100. The vehicle 100 may include a plurality of non-drive type LiDARs 330.

The LiDAR 330 may detect an object in a TOP manner or a phase-shift manner through the medium of a laser beam, and detect a position of the detected object, a distance from the detected object and a relative speed with the detected object.

The LiDAR 330 may be disposed on an appropriate position outside the vehicle for detecting an object located at the front, rear or side of the vehicle.

The ultrasonic sensor 340 may include ultrasonic wave transmitting and receiving portions. The ultrasonic sensor 340 may detect an object based on an ultrasonic wave, and detect a position of the detected object, a distance from the detected object and a relative speed with the detected object.

The ultrasonic sensor 340 may be disposed on an appropriate position outside the vehicle for detecting an object located at the front, rear or side of the vehicle.

The infrared sensor 350 may include infrared light transmitting and receiving portions. The infrared sensor 340 may detect an object based on infrared light, and detect a position of the detected object, a distance from the detected object and a relative speed with the detected object.

The infrared sensor 350 may be disposed on an appropriate position outside the vehicle for detecting an object located at the front, rear or side of the vehicle.

The processor 370 may control an overall operation of each unit of the object detecting apparatus 300.

The processor 370 may detect an object based on an acquired image, and track the object. The processor 370 may execute operations, such as a calculation of a distance from the object, a calculation of a relative speed with the object and the like, through an image processing algorithm.

The processor 370 may detect an object based on a reflected electromagnetic wave which an emitted electromagnetic wave is reflected from the object, and track the object. The processor 370 may execute operations, such as a calculation of a distance from the object, a calculation of a relative speed with the object and the like, based on the electromagnetic wave.

The processor 370 may detect an object based on a reflected laser beam which an emitted laser beam is reflected from the object, and track the object. The processor 370 may execute operations, such as a calculation of a distance from the object, a calculation of a relative speed with the object and the like, based on the laser beam.

The processor 370 may detect an object based on a reflected ultrasonic wave which an emitted ultrasonic wave is reflected from the object, and track the object. The processor 370 may execute operations, such as a calculation of a distance from the object, a calculation of a relative speed with the object and the like, based on the ultrasonic wave.

The processor may detect an object based on reflected infrared light which emitted infrared light is reflected from the object, and track the object. The processor 370 may execute operations, such as a calculation of a distance from the object, a calculation of a relative speed with the object and the like, based on the infrared light.

According to an embodiment, the object detecting apparatus 300 may include a plurality of processors 370 or may not include any processor 370. For example, each of the camera 310, the radar 320, the LiDAR 330, the ultrasonic sensor 340 and the infrared sensor 350 may include the processor in an individual manner.

When the processor 370 is not included in the object detecting apparatus 300, the object detecting apparatus 300 may operate according to the control of a processor of an apparatus within the vehicle 100 or the controller 170.

The object detecting apparatus 400 may operate according to the control of the controller 170.

The communication apparatus 400 is an apparatus for performing communication with an external device. Here, the external device may be another vehicle, or a server.

The communication apparatus 400 may perform the communication by including at least one of a transmitting antenna, a receiving antenna, and radio frequency (RF) circuit and RF device for implementing various communication protocols.

The communication apparatus 400 may include a short-range communication unit 410, a location information unit 420, a V2X communication unit 430, an optical communication unit 440, a broadcast transceiver 450 and a processor 470.

According to an embodiment, the communication apparatus 400 may further include other components in addition to the components described, or may not include some of the components described.

The short-range communication unit 410 is a unit for facilitating short-range communications. Suitable technologies for implementing such short-range communications include BLUETOOTH™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB (Wireless Universal Serial Bus), and the like.

The short-range communication unit 410 may construct short-range area networks to perform short-range communication between the vehicle 100 and at least one external device.

The location information unit 420 is a unit for acquiring position information. For example, the location information unit 420 may include a Global Positioning System (GPS) module or a Differential Global Positioning System (DGPS) module.

The V2X communication unit 430 is a unit for performing wireless communications with a server (Vehicle to Infra; V2I), another vehicle (Vehicle to Vehicle; V2V), or a pedestrian (Vehicle to Pedestrian; V2P). The V2X communication unit 430 may include an RF circuit implementing a communication protocol with the infra (V2I), a communication protocol between the vehicles (V2V) and a communication protocol with a pedestrian (V2P).

The optical communication unit 440 is a unit for performing communication with an external device through the medium of light. The optical communication unit 440 may include a light-emitting diode for converting an electric signal into an optical signal and sending the optical signal to the exterior, and a photodiode for converting the received optical signal into an electric signal.

According to an embodiment, a light-emitting unit may be integrally formed with lamps provided on the vehicle 100.

The broadcast transceiver 450 is a unit for receiving a broadcast signal from an external broadcast managing entity or transmitting a broadcast signal to the broadcast managing entity via a broadcast channel. The broadcast channel may include a satellite channel, a terrestrial channel, or both. The broadcast signal may include a TV broadcast signal, a radio broadcast signal and a data broadcast signal.

The processor 470 may control an overall operation of each unit of the communication apparatus 400.

According to an embodiment, the communication apparatus 400 may include a plurality of processors 470 or may not include any processor 470.

When the processor 470 is not included in the communication apparatus 400, the communication apparatus 400 may operate according to the control of a processor of another device within the vehicle 100 or the controller 170.

Meanwhile, the communication apparatus 400 may implement a display apparatus for a vehicle together with the user interface apparatus 200. In this instance, the display apparatus for the vehicle may be referred to as a telematics apparatus or an Audio Video Navigation (AVN) apparatus.

The communication apparatus 400 may operate according to the control of the controller 170.

The driving control apparatus 500 is an apparatus for receiving a user input for driving.

In a manual mode, the vehicle 100 may be operated based on a signal provided by the driving control apparatus 500.

The driving control apparatus 500 may include a steering input device 510, an acceleration input device 530 and a brake input device 570.

The steering input device 510 may receive an input regarding a driving (ongoing) direction of the vehicle 100 from the user. The steering input device 510 is preferably configured in the form of a wheel allowing a steering input in a rotating manner. According to some embodiments, the steering input device may also be configured in a shape of a touch screen, a touch pad or a button.

The acceleration input device 530 may receive an input for accelerating the vehicle 100 from the user. The brake input device 570 may receive an input for braking the vehicle 100 from the user. Each of the acceleration input device 530 and the brake input device 570 is preferably configured in the form of a pedal. According to some embodiments, the acceleration input device or the brake input device may also be configured in a shape of a touch screen, a touch pad or a button.

The driving control apparatus 500 may operate according to the control of the controller 170.

The vehicle operating apparatus 600 is an apparatus for electrically controlling operations of various devices within the vehicle 100.

The vehicle operating apparatus 600 may include a power train operating unit 610, a chassis operating unit 620, a door/window operating unit 630, a safety apparatus operating unit 640, a lamp operating unit 650, and an air-conditioner operating unit 660.

According to some embodiments, the vehicle operating apparatus 600 may further include other components in addition to the components described, or may not include some of the components described.

Meanwhile, the vehicle operating apparatus 600 may include a processor. Each unit of the vehicle operating apparatus 600 may individually include a processor.

The power train operating unit 610 may control an operation of a power train device.

The power train operating unit 610 may include a power source operating portion 611 and a gearbox operating portion 612.

The power source operating portion 611 may perform a control for a power source of the vehicle 100.

For example, upon using a fossil fuel-based engine as the power source, the power source operating portion 611 may perform an electronic control for the engine. Accordingly, an output torque and the like of the engine can be controlled. The power source operating portion 611 may adjust the engine output torque according to the control of the controller 170.

For example, upon using an electric energy-based motor as the power source, the power source operating portion 611 may perform a control for the motor. The power source operating portion 611 may adjust a rotating speed, a torque and the like of the motor according to the control of the controller 170.

The gearbox operating portion 612 may perform a control for a gearbox.

The gearbox operating portion 612 may adjust a state of the gearbox. The gearbox operating portion 612 may change the state of the gearbox into drive (forward) (D), reverse (R), neutral (N) or parking (P).

Meanwhile, when an engine is the power source, the gearbox operating portion 612 may adjust a locked state of a gear in the drive (D) state.

The chassis operating unit 620 may control an operation of a chassis device.

The chassis operating unit 620 may include a steering operating portion 621, a brake operating portion 622 and a suspension operating portion 623.

The steering operating portion 621 may perform an electronic control for a steering apparatus within the vehicle 100. The steering operating portion 621 may change a driving direction of the vehicle.

The brake operating portion 622 may perform an electronic control for a brake apparatus within the vehicle 100. For example, the brake operating portion 622 may control an operation of brakes provided at wheels to reduce speed of the vehicle 100.

Meanwhile, the brake operating portion 622 may individually control each of a plurality of brakes. The brake operating portion 622 may differently control braking force applied to each of a plurality of wheels.

The suspension operating portion 623 may perform an electronic control for a suspension apparatus within the vehicle 100. For example, the suspension operating portion 623 may control the suspension apparatus to reduce vibration of the vehicle 100 when a bump is present on a road.

Meanwhile, the suspension operating portion 623 may individually control each of a plurality of suspensions.

The door/window operating unit 630 may perform an electronic control for a door apparatus or a window apparatus within the vehicle 100.

The door/window operating unit 630 may include a door operating portion 631 and a window operating portion 632.

The door operating portion 631 may perform the control for the door apparatus. The door operating portion 631 may control opening or closing of a plurality of doors of the vehicle 100. The door operating portion 631 may control opening or closing of a trunk or a tail gate. The door operating portion 631 may control opening or closing of a sunroof.

The window operating portion 632 may perform the electronic control for the window apparatus. The window operating portion 632 may control opening or closing of a plurality of windows of the vehicle 100.

The safety apparatus operating unit 640 may perform an electronic control for various safety apparatuses within the vehicle 100.

The safety apparatus operating unit 640 may include an airbag operating portion 641, a seatbelt operating portion 642 and a pedestrian protecting apparatus operating portion 643.

The airbag operating portion 641 may perform an electronic control for an airbag apparatus within the vehicle 100. For example, the airbag operating portion 641 may control the airbag to be deployed upon a detection of a risk.

The seatbelt operating portion 642 may perform an electronic control for a seatbelt apparatus within the vehicle 100. For example, the seatbelt operating portion 642 may control passengers to be motionlessly seated in seats 110FL, 110FR, 110RL, 110RR using seatbelts upon a detection of a risk.

The pedestrian protecting apparatus operating portion 643 may perform an electronic control for a hood lift and a pedestrian airbag. For example, the pedestrian protecting apparatus operating portion 643 may control the hood lift and the pedestrian airbag to be open up upon detecting pedestrian collision.

The lamp operating unit 650 may perform an electronic control for various lamp apparatuses within the vehicle 100.

The air-conditioner operating unit 660 may perform an electronic control for an air conditioner within the vehicle 100. For example, the air-conditioner operating unit 660 may control the air conditioner to supply cold air into the vehicle when internal temperature of the vehicle is high.

The vehicle operating apparatus 600 may include a processor. Each unit of the vehicle operating apparatus 600 may individually include a processor.

The vehicle operating apparatus 600 may operate according to the control of the controller 170.

The operation system 700 is a system that controls various driving modes of the vehicle 100. The operation system 700 may operate in an autonomous driving mode.

The operation system 700 may include a driving system 710, a parking exit system 740 and a parking system 750.

According to embodiments, the operation system 700 may further include other components in addition to components to be described, or may not include some of the components to be described.

Meanwhile, the operation system 700 may include a processor. Each unit of the operation system 700 may individually include a processor.

According to embodiments, the operation system may be a sub concept of the controller 170 when it is implemented in a software configuration.

Meanwhile, according to embodiment, the operation system 700 may be a concept including at least one of the user interface apparatus 200, the object detecting apparatus 300, the communication apparatus 400, the vehicle operating apparatus 600 and the controller 170.

The driving system 710 may perform driving of the vehicle 100.

The driving system 710 may receive navigation information from a navigation system 770, transmit a control signal to the vehicle operating apparatus 600, and perform driving of the vehicle 100.

The driving system 710 may receive object information from the object detecting apparatus 300, transmit a control signal to the vehicle operating apparatus 600 and perform driving of the vehicle 100.

The driving system 710 may receive a signal from an external device through the communication apparatus 400, transmit a control signal to the vehicle operating apparatus 600, and perform driving of the vehicle 100.

The parking exit system 740 may perform an exit of the vehicle 100 from a parking lot.

The parking exit system 740 may receive navigation information from the navigation system 770, transmit a control signal to the vehicle operating apparatus 600, and perform the exit of the vehicle 100 from the parking lot.

The parking exit system 740 may receive object information from the object detecting apparatus 300, transmit a control signal to the vehicle operating apparatus 600 and perform the exit of the vehicle 100 from the parking lot.

The parking exit system 740 may receive a signal from an external device through the communication apparatus 400, transmit a control signal to the vehicle operating apparatus 600, and perform the exit of the vehicle 100 from the parking lot.

The parking system 750 may perform parking of the vehicle 100.

The parking system 750 may receive navigation information from the navigation system 770, transmit a control signal to the vehicle operating apparatus 600, and park the vehicle 100.

The parking system 750 may receive object information from the object detecting apparatus 300, transmit a control signal to the vehicle operating apparatus 600 and park the vehicle 100.

The parking system 750 may receive a signal from an external device through the communication apparatus 400, transmit a control signal to the vehicle operating apparatus 600, and park the vehicle 100.

The navigation system 770 may provide navigation information. The navigation information may include at least one of map information, information regarding a set destination, path information according to the set destination, information regarding various objects on a path, lane information and current location information of the vehicle.

The navigation system 770 may include a memory and a processor. The memory may store the navigation information. The processor may control an operation of the navigation system 770.

According to embodiments, the navigation system 770 may update prestored information by receiving information from an external device through the communication apparatus 400.

According to embodiments, the navigation system 770 may be classified as a sub component of the user interface apparatus 200.

The sensing unit 120 may sense a status of the vehicle. The sensing unit 120 may include a posture sensor (e.g., a yaw sensor, a roll sensor, a pitch sensor, etc.), a collision sensor, a wheel sensor, a speed sensor, a tilt sensor, a weight-detecting sensor, a heading sensor, a gyro sensor, a position module, a vehicle forward/backward movement sensor, a battery sensor, a fuel sensor, a tire sensor, a steering sensor by a turn of a handle, a vehicle internal temperature sensor, a vehicle internal humidity sensor, an ultrasonic sensor, an illumination sensor, an accelerator position sensor, a brake pedal position sensor, and the like.

The sensing unit 120 may acquire sensing signals with respect to vehicle-related information, such as a posture, a collision, an orientation, a position (GPS information), an angle, a speed, an acceleration, a tilt, a forward/backward movement, a battery, a fuel, tires, lamps, internal temperature, internal humidity, a rotated angle of a steering wheel, external illumination, pressure applied to an accelerator, pressure applied to a brake pedal and the like.

The sensing unit 120 may further include an accelerator sensor, a pressure sensor, an engine speed sensor, an air flow sensor (AFS), an air temperature sensor (ATS), a water temperature sensor (WTS), a throttle position sensor (TPS), a TDC sensor, a crank angle sensor (CAS), and the like.

The interface unit 130 may serve as a path allowing the vehicle 100 to interface with various types of external devices connected thereto.

Meanwhile, the interface unit 130 may serve as a path for supplying electric energy to a connected device. When the device is electrically connected to the interface unit 130, the interface unit 130 supplies electric energy supplied from a power supply unit to the device according to the control of the controller 170.

The memory 140 is electrically connected to the controller 170. The memory 140 may store basic data for units, control data for controlling operations of units and input/output data. The memory 140 may be a variety of storage devices, such as ROM, RAM, EPROM, a flash drive, a hard drive and the like in a hardware configuration. The memory 140 may store various data for overall operations of the vehicle 100, such as programs for processing or controlling the controller 170.

According to embodiments, the memory 140 may be integrated with the controller 170 or implemented as a sub component of the controller 170.

The controller 170 may control an overall operation of each unit of the vehicle 100. The controller 170 may be referred to as an Electronic Control Unit (ECU).

The power supply unit 860 may supply power required for an operation of each component according to the control of the controller 170. Specifically, the power supply unit 860 may receive power supplied from an internal battery of the vehicle, and the like.

At least one processor and the controller 170 included in the vehicle 100 may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro controllers, microprocessors, and electric units performing other functions.

What has been explained in relation to the vehicle 100 with reference to FIGS. 1 to 7 may be included in the following description of the vehicle 100. That is, the vehicle 100 related to the present invention may include at least one of the components explained with reference to FIGS. 1 to 7.

Hereinafter, an operational process for the vehicle 100 in accordance with an embodiment of the present invention to produce a driving stress map for each road section and recommend a suitable driving mode to the driver based on a stress level for a current road section will be described in details with reference to the accompanying drawings.

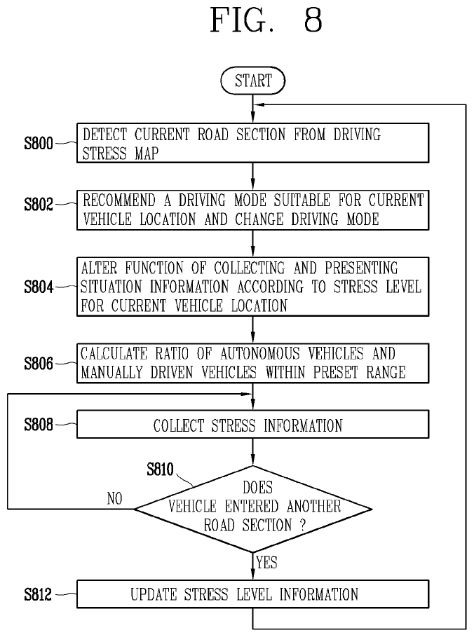

First of all, FIG. 8 is a flowchart showing an operational process for recommending a driving mode suitable for a current road section, in a vehicle in accordance with an embodiment of the present invention.

Referring to FIG. 8, the controller 170 of the vehicle 100 in accordance with the embodiment of the present invention may detect a current road section, from a stored driving stress map (S800).

Here, the driving stress map may be a map containing information on the driver's stress levels calculated for each road section. The stress level information may signify a score calculated from stress information collected from the driver when the vehicle is driving on each road section.

Also, the road section may be one of a plurality of sections into which each vehicle driving path, i.e., each road, included in the map is divided according to a set criterion. For example, each road may be defined as an area between preset landmarks (e.g., between two traffic light poles) or as a certain distance. That is, the driving stress map may signify map information created by mapping a different stress level to each road section, that is, each of the road sections into which a road is divided.

Meanwhile, the stress information may include the driver's biometric information collected while the vehicle is driving. Also, the stress information may be information related to the driver's behavior detected while the vehicle is driving. For example, the stress information may be information about the driver's heart rate or blood pressure collected while the vehicle is driving. Also, the stress information may be stress information related to the driver's specific behavior (e.g., honking the horn or speaking louder than a specific volume level).

The controller 170 then calculates stress scores according to each stress information and calculates the stress level for the road section the vehicle is driving on by putting the calculated stress scores together. As such, the controller 170 may map the stress level calculated for the road section the vehicle is currently driving on, and the driving stress map may be a map including at least one road section to which a stress level is mapped.

Accordingly, in the step S800, upon detecting a road section the vehicle 100 is currently driving on, from the driving stress map, the controller 170 may retrieve a stress level mapped to the detected road section. The controller 170 then may recommend to the user a driving mode suitable for the current vehicle location, i.e., the road section the vehicle is driving on, according to the retrieved stress level (S802).

Here, the step S802 may include a process of displaying notification information for indicating to the driver a driving mode deemed suitable for the current road section. Here, the controller 170 may determine a driving mode suitable for the road section the vehicle 100 is currently driving on, according to the stress level retrieved in the step S800.

For example, if the stress level retrieved in the step S800 exceeds a preset value (first reference value), the controller 170 may determine that the driver is under a lot of stress while driving on the road section where the vehicle 100 is currently located. As such, the controller 170 may determine that an autonomous driving mode is suitable for the driver on the current road section, and provide notification information for recommending autonomous driving mode. In this instance, the controller 170 may change the driving mode to autonomous driving mode based on what the driver selects after seeing the notification information.

In contrast, if the stress level retrieved in the step S800 is equal to or lower than the preset value, the controller 170 may determine that the driver is under little stress while driving on the road section where the vehicle 100 is currently located. As such, the controller 170 may determine that manual driving mode is suitable for the driver on the current road section, and provide notification information for recommending manual driving mode. In this instance, the controller 170 may change the driving mode to manual driving mode based on what the driver selects after seeing the notification information.

Meanwhile, in the step S802, the controller 170 may determine whether the driver is under severe stress when driving the vehicle on the road section where the vehicle 100 is currently located (e.g., whether the retrieved stress level exceeds a second reference value which is higher than the first reference value) according to the stress level retrieved in the step S800. In this case, it is obvious that the controller 170 may automatically change the driving mode of the vehicle 100 to autonomous driving mode.

On the contrary, the controller 170 may determine whether the driver is under very little stress when driving the vehicle on the road section where the vehicle 100 is currently located (e.g., whether the retrieved stress level is equal to or lower than a third reference value which is lower than the first reference value) according to the stress level retrieved in the step S800. In this case, it is obvious that the controller 170 may automatically change the driving mode of the vehicle 100 to manual driving mode.

To automatically change the driving mode, notification information for indicating a change of driving mode may be outputted so that the driver can be aware of this change. Also, when notification information related to the driving mode recommendation or automatic mode change is outputted, a preset audio signal or vibration, too, may be outputted to alert the driver.

Meanwhile, it is obvious that whether to output the notification information or not may be determined depending on the current driving mode of the vehicle 100. For example, in the step S802, the controller 170 may compare a driving mode deemed more suitable and the current driving mode of the vehicle 100, and output notification information for recommending a specific driving mode or indicating an automatic change to the specific driving mode only when the comparison result shows that the two driving modes are different.

That is, in a case where autonomous driving mode is deemed more suitable for the road section the vehicle 100 is currently driving on, according to the stress level retrieved in the step S800, if the vehicle 100 is already in autonomous driving mode, the controller 170 may not output notification information recommending a change to autonomous driving mode or indicating an automatic change to autonomous driving mode.

Meanwhile, it is obvious that, if the driver inputs a signal for changing the driving mode when the driving mode has been just changed based on the notification information or while the vehicle 100 is driving in a driving mode corresponding to the stress level mapped to the current road section, the controller 170 may output the notification information over again.

Meanwhile, once the driving mode is changed according to what the driver selects after seeing the notification information or according to an automatic change in the step S802, the controller 170 may alter a function of collecting and displaying information on situations around the vehicle 100 according to the stress level for the road section where the vehicle 100 is currently located (S804).

For example, the controller 170 may determine the picture quality of a black box or the resolution of captured images according to the stress level for the road section where the vehicle 100 is currently located. That is, the higher the stress level, the higher the picture quality of the black box or the resolution of captured images. In contrast, the lower the stress level, the lower the picture quality of the black box and the resolution of captured images. This is to obtain clear circumstantial evidence in case of accidents because a higher stress level leads to a higher risk of accidents.

Moreover, the controller 170 may determine the working range of V2X (vehicle-to-things) or V2V (vehicle-to-vehicle) according to the stress level for the road section where the vehicle 100 is currently located. For example, the controller 170 may increase the signal strength of V2X or V2V as the stress level becomes higher. In this case, strong signal strength enables a wider range of communication with vehicles or objects. Alternatively, the controller 170 may shorten the signal exchange cycle, in which V2X or V2V signals are exchanged, as the stress level becomes higher. This allows for more frequent exchange of signals with other vehicles or objects around the vehicle 100. Thus, it is possible to collect much denser information from other vehicles or objects around the vehicle 100.

In addition, the controller 170 may provide the driver with more information on road situations around the vehicle 100 as the stress level for the road section where the vehicle 100 is currently located becomes higher. In an example, the controller 170 may increase the number of displays 251 displaying information on road situations collected from around the vehicle 100. In an example, the controller 170 may output information on road situations detected from the rear side of the vehicle 100 through the displays 251 (e.g., clusters or CIDs (central information displays)) provided inside the vehicle.

In a case where such a large number of displays are used to output road situation information, the output of existing information may be restricted. That is, if the driving mode is automatically switched to autonomous driving mode due to a very high stress level, road situation information collected from around the vehicle 100 may be displayed in place of dashboard information outputted through the CIDs.

Meanwhile, if the driving mode is changed according to what the driver selects after seeing the notification information or according to an automatic change in the step S802, or if the vehicle 100 is driving in a driving mode corresponding to the stress level for the road section where the vehicle 100 is currently located, as selected by the driver, the controller 170 may identify the driving modes of other vehicles detected within a preset range. Also, the driver may be recommended to change to a driving mode based on the ratio of the identified driving modes of other vehicles (S806).

In the step S806, the controller 170 may detect the number of vehicles driving in autonomous driving mode among other vehicles located within the preset range. Also, the number of vehicles driving in manual driving mode among other vehicles located within the preset range may be detected. Also, the ratio of detected autonomous vehicles and manually driven vehicles may be calculated.

Meanwhile, the controller 170 may determine whether the ratio of vehicles driving in a specific driving mode is equal to or higher than a preset level, as a result of the radio calculation. If the ratio of vehicles driving in the specific driving mode is equal to or higher than the preset level, the specific driving mode and the current driving mode of the vehicle 100 may be compared with each other. Also, if the comparison result shows that the current driving mode of the vehicle 100 is not the specific driving mode, the controller 170 may recommend the driver to change the driving mode to the specific driving mode. In this case, the recommendation of a change of the driving mode may be made in a way similar to the way the notification information is outputted. In this instance, information on the calculated ratio may be provided to the driver. Also, if the driver chooses to change to the specific driving mode according to the recommendation information, the controller 170 may change the driving mode.

Next, the controller 170 may collect the driver's stress information (S808). As described above, the driver's biometric information and information related to the driver's behavior detected while driving may be collected as the stress information.

In an example, the controller 170 may check the driver's heart rate or blood pressure in the step S808. To this end, the controller 170 may be connected to a wearable device the driver is wearing to obtain the user's biometric information. In this case, the controller 170 may obtain the user's biometric information sensed from the wearable device.

Alternatively, the controller 170 may sense the driver's voice through a microphone or the like provided inside the vehicle 100. In this case, information related to the number of detections of voice with a preset volume or higher or the volume of voice may be collected as the stress information. Alternatively, the number of honks and the duration of horn honking may be collected as the stress information. Meanwhile, information on the driver's driving time may be collected as the stress information. This is because the driving activity itself may give the driver stress even if the driver does not honk the horn or raise his or her voice.

Meanwhile, the controller 170 may determine whether the vehicle 100 has entered another road section (S810). In an example, if the vehicle 100 gets closer to the boundary between the road section it is driving on and another road section, the controller 170 may determine that the vehicle 100 has entered the another road section. If the result of the determination in the step S810 shows that the vehicle 100 has not entered another road section, the controller 170 may return to the step S808 and collect stress information from the driver.

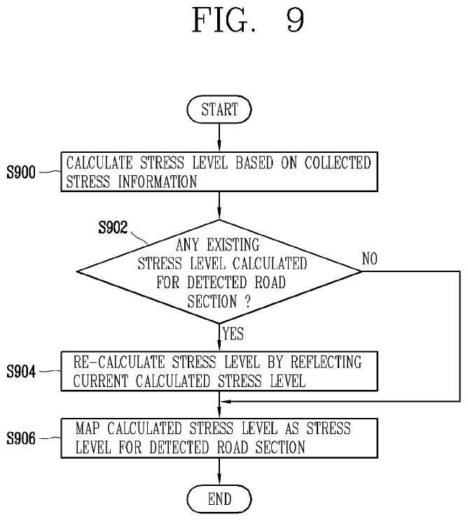

In contrast, if the result of the determination in the step S810 shows that the vehicle 100 has entered another road section (second road section), the controller 170 may update the stress level for the road section (first section) the vehicle 100 has just passed by based on the stress information collected so far (S812). In this case, the controller 170 may calculate the driver's stress scores based on the collected stress information, and calculate the stress score for the first section based on the calculated stress scores. Also, the stress level for the first road section may be updated based on the calculated stress score. An operational process for the step S812 of updating stress level will be described below in more details with reference to FIG. 9.

Meanwhile, once the update of the stress level for the first road section is completed in the step S812, the controller 170 may return to the step S800 and detect the stress level for the current road section—i.e., the second road section—from a driving stress map for the second road section. Then, the processes performed in the steps 802 through 812 may be repeated.

As described previously, the driving stress map may be a map containing stress levels calculated based on information collected when the driver is driving on each road section. Thus, it is obvious that the driving stress map may vary from driver to driver. In this case, different driving stress maps may have different stress levels for each road section. To this end, the memory 140 may store information on a plurality of driving stress maps, and the controller 170 may identify the driver of the vehicle 100 before the vehicle starts driving and load the driving stress map corresponding to the identified driver from the memory 140.

FIG. 9 is a flowchart showing an operational process for updating a stress level in a driving stress map based on stress information collected while driving, in a vehicle 100 in accordance with an embodiment of the present invention

Referring to FIG. 9, when the vehicle 100 enters another road section, the controller 170 of the vehicle 100 in accordance with the embodiment of the present invention may calculate a stress level based on the stress information collected in the step S808 of FIG. 8 (S900).

Here, the stress information may be biometric information such as the driver's heart rate or blood pressure. In this case, the controller 170 may retrieve a stress score corresponding to the measured heart rate or blood pressure from a preset stress score table. The stress score table may be a table containing stress scores corresponding to the driver's heart rates or blood pressures. Also, the higher the heart rate or blood pressure, the higher the stress score.

Moreover, the stress information may be information related to the driver's specific behavior (e.g., honking the horn or speaking louder than a specific volume level) collected while the vehicle is driving. In this case, the controller 170 may calculate a stress score based on the number of detections of the specific behavior and the duration of the specific behavior. In an example, if the driver honks the horn, a stress score corresponding to the horn honking may be retrieved, and the retrieved stress score may be increased depending on the duration of the horn honking. That is, the more often the horn is honked and the longer the horn is honked, the higher the stress score.

Meanwhile, the controller 170 may determine if the driver is driving recklessly. In an example, if the driver speeds up to over a certain level or the number of lane changes is a preset value or above, the controller 170 may determine that the driver is driving recklessly. In this case, the controller 170 may retrieve a stress score corresponding to the reckless driving. Also, the controller 170 may detect the driver's number of traffic light violations while driving. In this case, a traffic light violation may be regarded as reckless driving, and therefore a stress score corresponding to the number of traffic light violations may be calculated.

Once all of these stress scores are calculated based on their corresponding stress information, the controller 170 may put the calculated stress scores together and calculate the stress score for the first section. For example, the controller 170 may assign a weight to each of the stress scores, and add the weighted stress scores together. Then, the stress level for the first section may be calculated based on the total stress score.

Once the stress level for the first section is calculated, the controller 170 may check whether there is any existing stress level calculated for the first section (S902). If the check result shows that there is an existing stress level calculated for the first section, the current calculated stress level may be reflected to re-calculate the stress level (S904). For example, the controller 170 may calculate the average of the existing stress level calculated for the first section and the stress level calculated in the step S900. Then, the controller 170 may map the current calculated stress level as the stress level for the first section (S906).

If the result of the check in the step S902 shows that there is no stress level for the first section, the controller 170 may proceed immediately to the step S906 and map the current calculated stress level as the stress level for the first section.

This way, the controller 180 of the vehicle 100 in accordance with the embodiment of the present invention may collect the driver's biometric information and information related to the driver's behavior while the vehicle 100 is driving, and calculate the stress level for the road section the vehicle is currently driving on based on the collected information. Also, using map information including information (e.g., road information) about driving paths of the vehicle 100, the driving stress map may be created by mapping the calculated stress level to the road section the vehicle is currently driving on.

Meanwhile, the driving stress map may be created by mapping every corresponding driving mode according to a stress score mapped to each road section. For example, if the calculated stress level exceeds a preset, first reference value, autonomous driving mode may be mapped. In contrast, if the calculated stress level is equal to or lower than the first reference value, manual driving mode may be mapped. As such, the controller 170 may determine whether autonomous driving mode or manual driving mode is suitable for the current road section, based on the stress level calculated for the road section the vehicle 100 is driving on, that is contained in the driving stress map.

Meanwhile, it is obvious that the controller 170 may map a section where autonomous driving is necessary, depending on the stress level. For example, if a road section's stress level is calculated to exceed the first reference value and also exceed a second reference value, which is higher than the first reference value, the controller 170 may determine that autonomous driving mode is necessary on that road section. In this case, when the vehicle enters that road section, the controller 170 may output notification information indicating an automatic change to autonomous driving mode, instead of notification information recommending a change of the driving mode, in the step S802 of FIG. 8.

On the contrary, it is obvious that the controller 170 may map a section where manual driving is more recommendable, depending on the stress level. For example, if a road section's stress level is calculated to be equal to or lower than the first reference value and lower than a third reference value, which is lower than the first reference value, the controller 170 may determine that manual driving mode is necessary in that road section. In this case, when the vehicle enters that road section, the controller 170 may output notification information indicating an automatic change to manual driving mode, instead of notification information recommending a change of the driving mode, in the step S802 of FIG. 8.

Meanwhile, the stress level calculated for each road section, along with the type of the vehicle 100, may be transmitted to a preset server. The transmitted information may be used as information about stresses drivers feel on specific road sections depending on the type of vehicle 100. In this case, the transmitted information may be used for car manufacturers to improve parts of the vehicle 100.

Meanwhile, before the vehicle 100 enters a new road section, i.e., a second section, the controller 170 of the vehicle 100 in accordance with the embodiment of the present invention may present the driver with a driving mode suitable for the second section based on a stress level mapped to the second section. In this case, the controller 170 may output notification information for recommending the driving mode in advance before the vehicle 100 reaches the boundary of the current road section, that is, the first section. To this end, the controller 170 may set a handover zone for the current road section, and, if the vehicle 100 reaches the handover zone, may determine that the vehicle 100 is entering a new road section.

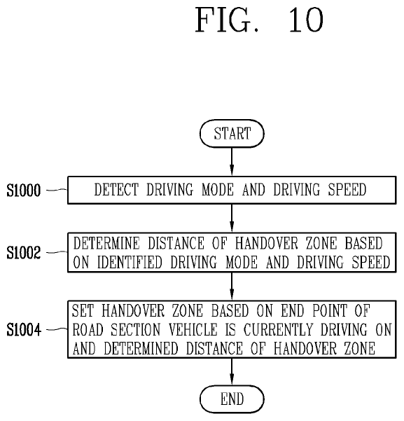

FIG. 10 is a flowchart showing an operational process for setting a handover zone for a current road section, in the controller 170 of a vehicle 100 in accordance with an embodiment of the present invention.

To set the handover zone, the controller 170 may identify the vehicle 100's driving mode and driving speed on the next road section (S1000). Then, the distance of the handover zone may be determined based on the identified driving mode and driving speed (S1002). Here, the driving mode on the next road section may correspond to the stress level for the next road section connecting to the road section where the vehicle 100 is currently located, depending on the direction of travel of the vehicle 100.

In this case, the controller 170 may vary the distance of the handover zone depending on the identified driving mode on the next road section. In an example, if the driving mode on the next road section is manual driving mode, the handover zone may be set longer than that for autonomous driving mode. Also, the distance of the handover zone may be increased as the driving speed of the vehicle 100 becomes faster. Also, the distance of the handover zone may vary depending on whether the next road section is a section where manual driving mode is recommended or a road section where the driving mode is automatically changed to manual driving mode.

Once the distance of the handover zone is determined in the step S1002, the controller 170 may set the handover zone based on the end point of the road section the vehicle 100 is currently driving on and the determined distance of the handover zone (S1004).

To this end, the controller 170 may determine the end point of the road section. Here, the end point of the road section may refer to the boundary of the road section corresponding to the direction of travel of the vehicle 100. Also, the controller 170 may define the handover zone as the distance determined in the step S1002 which extends backward from the determined end point of the road section along the road section.

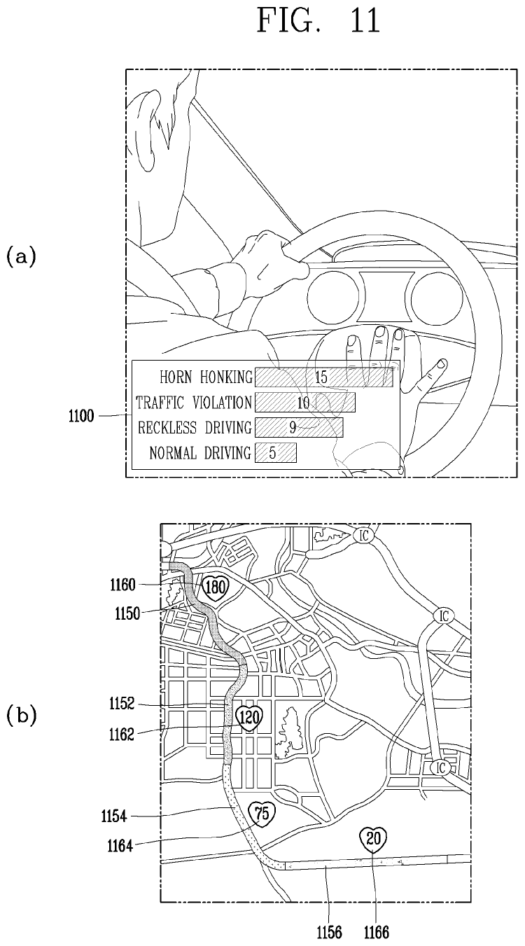

FIG. 11 is an illustration showing an example of collecting stress information from a driver and an example of a driving stress map to which calculated stress levels are mapped, in a vehicle in accordance with an embodiment of the present invention.

First of all, (a) of FIG. 11 shows an example 1100 in which stress information is acquired from the driver and a corresponding stress score is calculated. In this case, as shown in (a) of FIG. 11, a different stress score may be calculated for different stress information collected. In an example, if the driver honks the horn, a stress score may be calculated depending on the number of honks and the duration of the horn honking. Aside from this, a corresponding stress score may be calculated for a traffic violation or reckless driving. Besides, even if there is no specific behavior (normal driving), a stress score may be calculated depending on the driver's driving time

(b) of FIG. 11 shows an example of a driving stress map in accordance with an embodiment of the present invention. As shown in (b) of FIG. 11, a road the vehicle 100 is currently driving on may be divided into four road sections 1150, 1152, 1154, and 1156. In this case, stress levels 1160, 1162, 1164, and 1166 calculated for the road sections 1150, 1152, 1154, and 1156 may be matched to the road sections 1150, 1152, 1154, and 1156, respectively.

Meanwhile, information about a different driving mode may be mapped for each stress level. For example, the controller 170 may determine that autonomous driving mode is suitable for a stress level exceeding 100, and determine that manual driving mode is suitable for a stress level of 100 or lower. Also, the controller 170 may determine that autonomous driving mode is necessary for a stress level exceeding 150, and determine that manual driving mode is more recommendable for a stress level of 50 or lower.

In this case, if the vehicle 100 enters the first section 1150, the controller 170 may determine that autonomous driving mode is necessary according to the first stress level 1160 of “180” corresponding to the first section 1150. Accordingly, if the vehicle 100 enters the first road section 1150, the controller 170 may output notification information indicating an automatic change to autonomous driving mode.

On the one hand, if the vehicle 100 enters the second section 1152, the controller 170 may determine that autonomous driving mode is more suitable according to the second stress level 1162 of “120” corresponding to the second section 1152. Accordingly, if the vehicle 100 enters the second road section 1152, the controller 170 may output notification information indicating that autonomous driving mode is more suitable.

On the other hand, if the vehicle 100 enters the third section 1154, the controller 170 may determine that manual driving mode is more suitable according to the third stress level 1164 of “75” corresponding to the third section 1154. Accordingly, if the vehicle 100 enters the third road section 1154, the controller 170 may output notification information indicating that manual driving mode is more suitable.

On the other hand, if the vehicle 100 enters the fourth section 1156, the controller 170 may determine that manual driving mode is more recommendable according to the fourth stress level 1166 of “20” corresponding to the fourth section 1156. Accordingly, if the vehicle 100 enters the fourth road section 1156, the controller 170 may output notification information indicating an automatic change to manual driving mode.

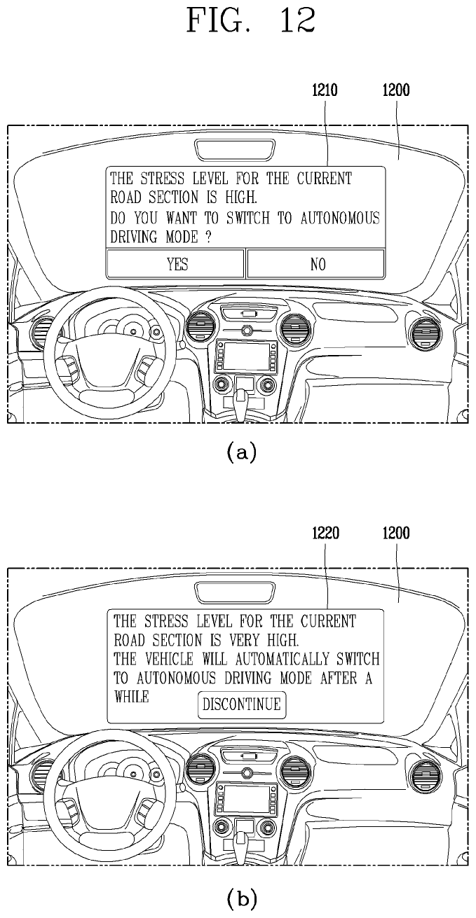

FIG. 12 is an illustration showing an example in which a driver is recommended and forced to switch to autonomous driving mode, in a vehicle 100 in accordance with an embodiment of the present invention.

First of all, (a) of FIG. 12 shows an example in which a stress level for a road section the vehicle 100 is currently driving on exceeds a preset, first reference value. In this instance, as described above, the controller 170 may determine that autonomous driving mode is more suitable for the current road section, and output notification information 1210 for recommending the driver to change to autonomous driving mode.

The notification information 1210 may be information that guides the driver to change the driving mode as they choose. That is, as shown in (a) of FIG. 12, if the driver selects “Yes” in response to the notification information 1210, the driving mode of the vehicle 100 may be changed to autonomous driving mode.

In contrast, (b) of FIG. 12 shows an example in which a stress level for the road section the vehicle 100 is currently driving on exceeds a second reference value which is higher than the preset, first reference. In this instance, as described above, the controller 170 may determine that autonomous driving mode is necessary for the current road section. Then, the controller 170 may output notification information 1220 for indicating an automatic change to autonomous driving mode.

In this case, the notification information 1220 may be information indicating that the driving mode will be automatically changed to autonomous driving mode after a given amount of time. That is, as shown in (b) of FIG. 12, if the driver does not choose to discontinue the change upon seeing the notification information 1220, the driving mode may be automatically changed to autonomous driving mode.

Although the foregoing description has been given of a change to autonomous driving mode according to an existing calculated stress level, it is obvious that an automatic change to autonomous driving mode may be made based on biometric information acquired from the driver and situations around the vehicle 100

In an example, even when the vehicle 100 is driving in manual driving mode, the controller 170 may recommend a change to autonomous driving mode through indication information if the driver is under a lot of stress or the driver's health condition is worsening based on the biometric information acquired from the driver. Alternatively, if the driver's health condition is worsening or the driver is under a lot of stress—for example, the driver's heart rate or blood pressure is a preset level or above, it is obvious that, upon detecting an object around the vehicle 100 whose possibility of collision is more than a certain level, the driver may be forced to switch to autonomous driving mode and then drive around the object.

Besides, it is obvious that the controller 170 of the vehicle 100 in accordance with the embodiment of the present invention may restrict some of the functions of the vehicle 100 based on a biometric signal detection result. In an example, if the driver's heart rate or blood pressure is a preset level or above, the controller 170 may restrict the vehicle from speeding up to over a certain level or from changing lanes.

Meanwhile, it is obvious that the controller 170 of the vehicle 100 may control the vehicle's air conditioning system based on the biometric signal detection result. For example, the controller 170 may ventilate the air or adjust the angle of the seat backrest. Also, the seat height may be adjusted relative to the driver's eye height.

Moreover, the controller 170 may output a preset image for relaxing the driver's mind and body based on the biometric signal detection result. In this case, the preset image is an image the driver sets in advance, which may be a family photo or pet photo. Also, questions for checking the driver's health condition may be outputted to prevent driver drowsiness or check the driver's health condition. Besides, it is obvious that, if the biometric signal detection result shows that the driver is in a risky health condition, the controller 170 may make an emergency call to a preset number.

Meanwhile, it is obvious that the controller 170 of the vehicle 100 in accordance with the embodiment of the present invention may recommend a lower-stress path among a number of paths to a destination, based on the driver's biometric information detected. For example, the controller 170 may add together the stress levels for all road sections of each path to the destination, based on the stress levels for the road sections included in the driving stress map. Then, the path with the lowest total stress level may be recommended to the driver.

While the foregoing description has been given on the assumption that a driving stress map containing stress level information on a road section the vehicle is currently driving is stored, it is obvious that the driving stress map may not be stored or the stress level information for that road section may not be contained in the driving stress map. For example, a stress level for a road section the driver drives on for the first time may not be included.

In this case, it is needless to say that the controller 170 of the vehicle 100 in accordance with the embodiment of the present invention may acquire stress level information from other drivers for the current road section. For example, if there is a map containing a stress level for the current road section, among other driving stress maps stored in the memory 140, the stress level information contained in the driving stress map may be used. Alternatively, stress level information may be collected from other vehicles around via V2V communication. In this case, the stress level for the current road section may be calculated by averaging the collected stress level information.

Alternatively, the controller 170 may create stress level information based on information on the features and type of a road section. In an example, a stress level may be calculated based on the number of curves or the slope of a road section. Alternatively, stress level information may be created based on the number of traffic accidents that occurred during a given period.

While the foregoing description has been given of an example in which the controller 170 of the vehicle 100 performs the above-described operations of the present invention, the above-described operations of the present invention may be performed by a vehicle control device connected to the controller 170 of the vehicle 100. In this case, the above-described operations of the present invention may be performed by a processor of the vehicle control device. In this case, stress level information and a driving stress map including road sections to which information on driving modes corresponding to different stress levels is mapped may be provided in a memory of the vehicle control device.

The present invention can be implemented as computer-readable codes in a program-recorded medium. The computer-readable medium may include all types of recording devices each storing data readable by a computer system. Examples of such computer-readable media may include hard disk drive (HDD), solid state disk (SSD), silicon disk drive (SDD), ROM, RAM, CD-ROM, magnetic tape, floppy disk, optical data storage element and the like. Also, the computer-readable medium may also be implemented as a format of carrier wave (e.g., transmission via an Internet). The computer may include the processor or the controller. Therefore, it should also be understood that the above-described embodiments are not limited by any of the details of the foregoing description, unless otherwise specified, but rather should be construed broadly within its scope as defined in the appended claims. Therefore, all changes and modifications that fall within the metes and bounds of the claims, or equivalents of such metes and bounds are therefore intended to be embraced by the appended claims.

1. A vehicle control device for controlling a vehicle, comprising:
a memory that stores a driving stress map containing stress level information which is calculated for each road section based on a driver's stress information collected while the vehicle is driving on each road section; and
a processor that retrieves a stress level for a road section where the vehicle is currently located from the driving stress map and outputs notification information recommending a change to a first driving mode or second driving mode according to the retrieved stress level.

2. The vehicle control device of claim 1, wherein the processor controls the vehicle to output first notification information recommending a change to the first driving mode or second driving mode, based on whether the stress level for the current location of the vehicle exceeds a preset, first value.

3. The vehicle control device of claim 2, wherein, if the stress level for the current location of the vehicle exceeds the preset, first reference value and also exceeds a second reference value, which is higher than the first reference value, the processor controls the vehicle to output second notification information indicating an automatic change to the first driving mode, and, if the stress level for the current location of the vehicle is equal to or lower than the first reference value and lower than a third reference value, which is lower than the first reference value, the processor controls the vehicle to output third notification information indicating an automatic change to the second driving mode.

4. The vehicle control device of claim 1, wherein the processor collects the stress information such as the driver's biometric information acquired for the road section the vehicle is currently driving on and information related to the driver's specific behavior detected while the vehicle is driving.

5. The vehicle control device of claim 4, wherein the processor detects whether the vehicle enters a second road section which is different from a first road section the vehicle is currently driving on, and calculates a stress score from stress information collected for the first road section according to the detection result and updates an existing stress level calculated for the first road section.

6. The vehicle control device of claim 5, wherein, if the vehicle enters a handover zone set for the first road section, the processor detects that the vehicle is entering the second road section, retrieves a stress level for the second road section from the driving stress map, and outputs the notification information according to the retrieved stress level.

7. The vehicle control device of claim 6, wherein the processor varies the distance of the handover zone based on a driving mode suitable for the stress level for the second road section and the driving speed of the vehicle.

8. The vehicle control device of claim 1, wherein the processor controls the vehicle to alter a function of collecting and displaying information on situations around the vehicle based on the stress level for the road section where the vehicle is currently located, the stress level being retrieved from the driving stress map.

9. The vehicle control device of claim 8, wherein the processor controls the vehicle to change the picture quality of a black box or the resolution of captured images based on the retrieved stress level or to change the strength or exchange cycle of communication signals for V2X (vehicle-to-things) or V2V (vehicle-to-vehicle).

10. The vehicle control device of claim 8, wherein, if the retrieved stress level is higher than a preset level, the processor controls the vehicle to display road situation information collected from around the vehicle, in place of dashboard information outputted through CIDs (central information displays).

11. The vehicle control device of claim 1, wherein the processor calculates the ratio of autonomous vehicles and manually driven vehicles to other vehicles located within a preset range from the vehicle, and, if the calculation result shows that the ratio of vehicles driving in a specific driving mode is equal to or higher than a preset value, compares the specific driving mode and the driving mode of the vehicle and controls the vehicle to output notification information recommending a driving mode change to the specific driving mode according to the comparison result.

12. The vehicle control device of claim 1, wherein the processor controls the vehicle to output the notification information according to a result of comparing a driving mode suitable for the stress level for the road section where the vehicle is currently located and the current driving mode of the vehicle, the stress level being retrieved from the driving stress map, and the current driving mode of the vehicle.

13. The vehicle control device of claim 1, wherein, when the vehicle is driving in manual driving mode, the processor controls the vehicle to output notification information recommending a change to autonomous driving mode based on a result of sensing the driver's biometric information.

14. The vehicle control device of claim 13, wherein, when the vehicle is driving in manual driving mode, the processor controls the vehicle in such a way that the driver is forced to switch to autonomous driving mode and drive around an object detected from around the vehicle based on a result of sensing the driver's biometric information and the possibility of colliding the object.

15. The vehicle control device of claim 1, wherein, when the vehicle is driving in manual driving mode, the processor controls the vehicle in such a way that at least one of the vehicle's functions is restricted based on a result of sensing the driver's biometric information,
wherein the restricted vehicle function involves speeding up to over a certain speed and changing lanes.

16. A control method for a vehicle control device for controlling a vehicle, the control method comprising:
a first step of retrieving a stress level for a road section the vehicle is driving on from a driving stress map, the driving stress map containing stress level information which is calculated for each road section based on a driver's stress information collected while the vehicle is driving on each road section;
a second step of determining whether a driving mode suitable for the road section the vehicle is currently driving on is autonomous driving mode or manual driving mode, based on the retrieved stress level;
a third step of determining whether an automatic change to the driving mode determined in the second step is necessary, based on the retrieved stress level; and
a fourth step of outputting notification information recommending a change to a specific driving mode or notification information indicating a change to the specific driving mode, according to the result of the determination in the third step.
